(19) United States
(12) Patent Application Publication
(10) Pub. No.: US 2021/0207078 A1
(43) Pub. Date: Jul. 8, 2021

(54) BIOSYNTHESIS AND RECOVERY OF SECONDARY METABOLITES

(71) Applicant: MANUS BIO, INC., Cambridge, MA (US)

(72) Inventors: Aaron LOVE, Cambridge, MA (US); Adel GHADERI, Cambridge, MA (US); Jason Eric DONALD, Cambridge, MA (US); Christine Nicole S. SANTOS, Cambridge, MA (US); Ajikumar Parayil KUMARAN, Cambridge, MA (US)

(21) Appl. No.: 17/151,871

(22) Filed: Jan. 19, 2021

Related U.S. Application Data
(63) Continuation-in-part of application No. PCT/US2019/054703, filed on Oct. 4, 2019.
(60) Provisional application No. 62/741,840, filed on Oct. 5, 2018.

(51) Int. Cl. C12N 1/20; C12N 1/16; C12N 1/26; C12P 5/00

(52) U.S. Cl. CPC C12N 1/20; C12P 5/007; C12N 1/26; C12N 1/16

(57) ABSTRACT

Aspects of the invention provide methods for producing one or more secondary metabolites from microbial culture. In various embodiments, the method comprises culturing a microbial cell producing a secondary metabolite for recovery from a bioreactor medium, the medium comprising an aqueous phase and an extraction phase. The composition of the extraction phase, and the relevant amount with respect to the aqueous phase, enhances production of the secondary metabolite from microbial cells and/or enhances extracellular transfer of the metabolite.

### PRIORITY

This Application is a continuation-in-part of PCT/US2019/054703, filed Oct. 4, 2019, which claims priority to and the benefit of U.S. Provisional Application No. 62/741,840, filed Oct. 5, 2018, each of which is hereby incorporated by reference in its entirety.

### BACKGROUND

Production of natural products often involves extraction from natural sources, which sometimes produce the compound of interest at low or even trace amounts. These extraction methods typically offer low yield, are not amenable to large-scale production, and are generally not sustainable. Metabolic engineering of microorganisms for production of natural compounds can potentially provide high yields of these products from cheap carbon sources or cheap and abundant precursors.

However, many natural products are volatile organic compounds susceptible to evaporation and air stripping, are toxic to microbial cells, and/or can be poorly soluble in aqueous solution if secreted into the fermentation medium. The product can therefore be lost during fermentation, e.g., by evaporation, air stripping, or otherwise difficult to produce or recover efficiently. One approach is the use of a compound that is immiscible with the aqueous fermentation medium and that can act as an extractant for the desired product. The target product partitions between the extractant and the aqueous fermentation medium, thereby sequestering the product in an organic phase. A 10% dodecane overlayer has conventionally been used for this purpose, with the assumption that low toxicity to the microorganism and low emulsion-forming organic phases were the desired properties for the organic phase. The performance of various extraction phases for organic product production via fermentation has been sparsely investigated.

### SUMMARY OF THE INVENTION

Aspects of the invention provide methods for producing one or more secondary metabolites from microbial culture, e.g., in a bioreactor. In various embodiments, the method comprises culturing a microbial cell producing a secondary metabolite for recovery from a bioreactor medium, the medium comprising an aqueous phase and an extraction phase. The composition of the extraction phase, and the relevant amount with respect to the aqueous phase, enhances production of the secondary metabolite from microbial cells and/or enhances extracellular transfer of the metabolite.

Microbial production of natural products relies on product transport to the extracellular environment, where the extracellular milieu should prevent product degradation, evaporation, and provide ease of separation and recovery. Dodecane has typically been employed for this purpose. Specifically, a 10% dodecane overlay has been used throughout industrial and academic experiments when conducting microbial fermentations of volatile natural products.

In accordance with aspects of the invention, an extraction phase can be designed to enhance production of a secondary metabolite from microbial fermentations. Without wishing to be bound by theory, extraction phase phenomena (including emulsion properties) that may contribute to enhanced production may include: facilitation of mass transfer between the intracellular and extracellular environments; effects on product export from the cell, or selective product export over precursors; oxygen/gas transfer to and from the bulk aqueous phase of the fermentation; and effects on cell membrane composition and cell metabolism.

For example, the composition and relative amount of the extraction phase may enhance production of the metabolite from microbial cells and/or enhance extracellular transfer of the metabolite as compared to a 10% (v/v) overlayer of dodecane. That is, a 10% overlayer of dodecane can be employed as a comparator, where the selected extraction phase will perform significantly better in terms of product biosynthesis and/or recovery.

In some embodiments, the composition of the extraction phase and relative amount of the extraction phase relative to the aqueous phase (% vol.) produce at least a 10% increase in the amount of the secondary metabolite in the extraction phase, as compared to a 10% (v/v) overlayer of dodecane employed under the same conditions. In some embodiments, the composition of the extraction phase and relative amount of the extraction phase relative to the aqueous phase (% vol.) produce at least a 20% increase in the amount of the secondary metabolite in the extraction phase, as compared to a 10% (v/v) overlay of dodecane employed under the same conditions.

In accordance with various embodiments, the method will employ an extraction phase that is less than 10% (v/v) relative to the aqueous phase. As demonstrated herein, a significantly lower amount of the extraction phase relative to the aqueous phase can actually enhance product yield, suggesting that the extraction phase impacts more than simply product sequestration. Accordingly, in some embodiments, the extraction phase is employed at from about 0.1% to about 8% (v/v) with respect to the aqueous phase, or the extraction phase is employed at from about 0.5% to about 5% (v/v) with respect to the aqueous phase, or the extraction phase is employed at about 1% to about 3% (v/v) with respect to the aqueous phase.

In some embodiments, the amount of the extraction phase in the bioreactor relative to the aqueous phase (% vol.) is the relative amount that provides the maximum yield of the secondary metabolite, within 10%. For example, the relative amount of the extraction phase relative to the aqueous phase can be varied, and evaluated for the peak in production of the secondary metabolite under particular production conditions.

In various embodiments, the method can be employed at various scales, including pilot scale and large commercial scale.

In various embodiments, the method may be employed for production of secondary metabolites using any microbial system, including but not limited to bacteria and yeast.

The method can be employed for the production of various types of secondary metabolites, which can be natural products of the microbial cell, or products produced by heterologous expression of enzymes. In some embodiments, the secondary metabolite is a plant product, produced in bacteria or yeast through heterologous enzyme expression. In various embodiments, the secondary metabolite is a terpene, terpenoid, alkaloid, cannabinoid, steroid, saponin, glycoside, stilbenoid, polyphenol, flavonoid, antibiotic, polyketide, fatty acid, or peptide.

The microbial cell is grown in an aqueous phase in a bioreactor, and may be cultured in batch culture, continuous culture, or semi-continuous culture. In some embodiments, the microbial cell is cultured using a fed-batch process comprising a first phase where bacterial biomass is created, followed by a secondary metabolite production phase. The aqueous phase generally comprises an appropriate cell culture medium, and may further comprise precursor molecules for production of the secondary metabolite. In some embodiments, carbon substrates are fed to the culture for production of the target product. In other embodiments, the microbial cells are fed product precursors, which may be substrates for synthetic enzymes, and/or substrates for glycosylation, oxygenation, or prenylation, or transfer of other chemical groups or moieties to a core structure.

The extraction phase is added to the culture, at least during the biosynthesis phase, and can be an organic overlayer that sequesters the secondary metabolite for recovery, in addition to enhancing biosynthesis and extracellular transport. The extraction phase is predominately composed of substantially non-volatile compounds at bioreactor conditions. Components of the extraction phase will generally be liquid under fermentation conditions, and have a boiling point above about 150° C.

In some embodiments, the extraction phase comprises (or predominately comprises) one or more members selected from: medium, long chain, or cyclic hydrocarbon(s); plant or vegetable oil or components thereof; fatty acid glyceride(s) (e.g., triglycerides), and fatty acid ester(s). Extraction phases can comprise or further comprise one or a blend of alkanes, one or a blend of ionic liquids, one or a blend of silicon oils, one or a blend of perfluorinated oils, and one of a blend of fatty acids, any of which may be stabilized by surfactant(s).

In some embodiments, the extraction phase comprises or predominately comprises one or more plant oils or vegetable oils. In some embodiments, the extraction phase comprises safflower oil.

When using extraction phases of less than 10% (v/v) with respect to the aqueous phase, after recovery of the extraction phase, the extraction phase can contain a high mass of the secondary metabolite (the product(s)). In some embodiments, the mass of product recovered is higher than with the use of a 10% dodecane overlayer. In various embodiments, after the production phase of the culture, the secondary metabolite is at least about 10% of the extraction phase by weight, or is at least about 20% of the extraction phase by weight, or is at least about 50% of the extraction phase by weight.

The secondary metabolite is recovered from the extraction phase, and product optionally isolated by any suitable process. In some embodiments, the product is purified by sequential extraction and purification. For example, the product may be purified by chromatography-based separation and recovery and/or distillation.

In various embodiments, the recovered secondary metabolite product is incorporated into a consumer or industrial product. For example, the product may be a flavor product, a fragrance product, a sweetener, a pharmaceutical, a dietary supplement, a cosmetic (including skin or hair care product), a cleaning product, a detergent or soap, or a pest control product.

Aspects and embodiments will be further apparent in accordance with the following detailed description.

### DESCRIPTION OF THE FIGURES

FIG. 1 shows that valencene titers vary with different extraction phases. A small scale 96 well plate fermentation was carried out with a strain producing valencene and oxygenated valencene. Only the non-oxygenated valencene titers are shown.

FIGS. 2A and B show that lower safflower oil percentage improves both overall fermentation productivity as well as conversion to oxygenated products in both shake flask and 96-well plate fermentations.

FIG. 3 shows 2 L bioreactor data comparing 10% and 1% safflower oil extractive phase. The results demonstrate an increase in the % oxygenated conversion with an increase in overall titer. The total oxygenated production with 1% safflower extractive phase is significantly higher. Microbial growth is similar under the two conditions.

### DETAILED DESCRIPTION OF THE INVENTION

Aspects of the invention provide methods for producing one or more secondary metabolites from microbial culture, e.g., in a bioreactor. In various embodiments, the method comprises culturing a microbial cell producing a secondary metabolite for recovery from a bioreactor medium, the medium comprising an aqueous phase and an extraction phase. The composition and relative amount of the extraction phase, with respect to the aqueous phase, enhances production of the secondary metabolite from microbial cells and/or enhances extracellular transfer of the metabolite.

Microbial production of natural products relies on product transport to the extracellular environment, where the extracellular milieu should prevent product degradation, evaporation, and provide ease of separation and recovery. Dodecane has typically been employed for this purpose. Specifically, a 10% dodecane overlay has been used throughout industrial and academic experiments when conducting microbial fermentations of volatile natural products. As used herein, the term “fermentation” refers to the bulk growth of microorganisms in a growth medium with the goal of producing a chemical product. The chemical product is referred to herein as a “secondary metabolite.” Secondary metabolites are organic compounds that are not directly involved in the normal growth, development, or reproduction of the host. In some embodiments, the biosynthesis of the secondary metabolite is the result of one or more recombinant enzymes. In some embodiments, the secondary metabolite is a natural product of a plant species, or a derivative of a natural product from a plant species.

In accordance with aspects of the invention, an extraction phase can be designed to enhance production of a secondary metabolite from microbial fermentations. Without wishing to be bound by theory, extraction phase phenomena (including emulsion properties) that may contribute to enhanced production may include: facilitation of mass transfer between the intracellular and extracellular environments; effects on product export from the cell, or selective product export over precursors; impacts on oxygen/gas transfer to and from the bulk aqueous phase of the fermentation; and effects on cell membrane composition and cell metabolism.

For example, the composition and relative amount of the extraction phase may enhance production of the metabolite from microbial cells and/or enhance extracellular transfer of the metabolite as compared to a 10% (v/v) overlayer of dodecane. That is, a 10% overlayer of dodecane can be employed as a comparator, where the selected extraction phase will perform significantly better in terms of product biosynthesis and/or recovery.

In some embodiments, the composition of the extraction phase and relative amount of the extraction phase relative to the aqueous phase (% vol.) produce at least a 10% increase in the amount of the secondary metabolite in the extraction phase, as compared to a 10% (v/v) overlayer of dodecane employed under the same conditions. In some embodiments, the composition of the extraction phase and relative amount of the extraction phase relative to the aqueous phase (% vol.) produce at least a 20% increase in the amount of the secondary metabolite in the extraction phase, as compared to a 10% (v/v) overlay of dodecane employed under the same conditions.

In accordance with various embodiments, the method will employ an extraction phase that is less than 10% (v/v) relative to the aqueous phase. As demonstrated herein, a significantly lower amount of the extraction phase relative to the aqueous phase can actually enhance product yield, suggesting that the extraction phase impacts more than simply product sequestration. In some embodiments, the composition of the extraction phase improves yield of the secondary metabolite at about 8% (v/v) with respect to the aqueous phase, or at about 5% (v/v) with respect to the aqueous phase, or at about 3% (v/v) with respect to the aqueous phase, or at about 1% (v/v) with respect to the aqueous phase, as compared to 10% (v/v) of the same extraction phase with respect to the aqueous phase. Accordingly, in some embodiments, the extraction phase is employed at from about 0.1% to about 8% (v/v) with respect to the aqueous phase, or the extraction phase is employed at from about 0.5% to about 5% (v/v) with respect to the aqueous phase, or the extraction phase is employed at about 1% to about 3% (v/v) with respect to the aqueous phase.

In some embodiments, the amount of the extraction phase in the bioreactor relative to the aqueous phase (% vol.) is the relative amount that provides the maximum yield of the secondary metabolite, within 10%. For example, the relative amount of the extraction phase relative to the aqueous phase can be varied, and evaluated for the peak in production of the secondary metabolite under particular production conditions. The relative amount of the extraction phase that maximizes yield at the selected culture conditions (within 10%), is selected for production.

In various embodiments, the method can be employed at various scales, including pilot scale and large commercial scale. For examples, the volume of the aqueous phase in the bioreactor can be at least about 2 L, or at least about 10 L, or at least 100 L, or at least about 200 L, or at least about 500 L, or at least about 1,000 L, or at least about 10,000 L, or at least about 100,000 L, or at least about 500,000 L. In some embodiments, the bioreactor is a stirred tank bioreactor. In some embodiments, the culture is from about 300 L to about 1,000,000 L.

Comparisons of product titer between different extraction phase compositions and relative amounts can be evaluated at commercial production conditions, or in some embodiments, evaluated at peak microbial growth (before stationary phase) using a 2 L stirred tank bioreactor. In some embodiments, comparisons of product titer with dodecane (e.g., 10% dodecane relative to the aqueous phase) are conducted at peak microbial growth (before stationary phase) in a 2 L stirred tank bioreactor.

In various embodiments, the method may be employed for production of secondary metabolites using any microbial system, including but not limited to bacteria and yeast. In some embodiments, the microbe is a bacterium, and may be of a genus selected from Escherichia, Bacillus, Corynebacterium, Rhodobacter, Zymomonas, Vibrio, Pseudomonas, Agrobacterium, Brevibacterium, and Paracoccus. In some embodiments, the bacterium is a species selected from Escherichia coli, Bacillus subtilis, Corynebacterium glutamicum, Rhodobacter capsulatus, Rhodobacter sphaeroides, Zymomonas mobilis, Vibrio natriegens, or Pseudomonas putida. In some embodiments, the bacterium is E. coli. In various embodiments, the microbial cell is a yeast cell, which is a species of Saccharomyces, Pichia, or Yarrowia. For example, the microbial cell may be a species selected from Saccharomyces cerevisiae, Pichia pastoris, and Yarrowia lipolytica. In some embodiments, the yeast cell is an oleaginous yeast, such as Yarrowia lipolytica.

The method can be employed for the production of various types of secondary metabolites, which can be natural products of the microbial cell, or products produced by heterologous expression of enzymes. In some embodiments, the secondary metabolite is a plant product, produced in bacteria or yeast through heterologous enzyme expression. In various embodiments, the secondary metabolite is a terpene, terpenoid, alkaloid, cannabinoid, steroid, saponin, glycoside, stilbenoid, polyphenol, flavonoid, antibiotic, polyketide, fatty acid, or peptide.

In exemplary embodiments, the secondary metabolite comprises a terpene or terpenoid (or “isoprenoid”). Terpenes and terpenoids, and enzymatic pathways, are described for example in U.S. Pat. No. 8,927,241, which is hereby incorporated by reference in its entirety. IPP and DMAPP are the precursors of terpenes and terpenoids, including monoterpenoids, sesquiterpenoids, diterpenoids, and triterpenoids, which have particular utility in the flavor, fragrance, cosmetics, and food sectors. Synthesis of terpenes and terpenoids proceeds via conversion of IPP and DMAPP precursors to geranyl diphosphate (GPP), farnesyl diphosphate (FPP), or geranylgeranyl diphosphate (GGPP), through the action of a prenyl transferase enzyme (e.g., GPPS, FPPS, or GGPPS). Such enzymes are known, and are described for example in U.S. Pat. No. 8,927,241, WO 2016/073740, and WO 2016/029153, which are hereby incorporated by reference in their entireties. There are two major biosynthetic routes for the essential isoprenoid precursors isopentenyl diphosphate (IPP) and dimethylallyl diphosphate (DMAPP), the mevalonate (MVA) pathway and the methylerythritol phosphate (MEP) pathway. The MVA pathway is found in most eukaryotes, archaea and a few eubacteria. The MEP pathway is found in eubacteria, the chloroplasts of plants, cyanobacteria, algae and apicomplexan parasites. E. coli and other Gram-negative bacteria utilize the MEP pathway to synthesize IPP and DMAPP metabolic precursors.

Exemplary terpene and terpenoid products include (−)-khusimone, (−)-limonene, (−)-methyl-(1R,2R,5S)-khusimal, (−)-methyl-(1R,2S,5S)-khusimal, (−)-rotundone, (+)-aromadendrene, (+)-khusimone, (+)-limonene, (+)-nootkatone, (1R,2R,5S)-khusimal, (1R,2S,5S)-khusimal, 1,4-cineole, 10-epi-gamma-eudesmol, 4-carvomenthenol, 4-terpineol, abietadiene, abietic acid, acetyl beta-caryophyllene, agarofuran, agarospirol, alpha pinene, alpha-bisabolol, alpha-cedrene, alpha-copaene, alpha-copaene-11-ol, alpha-damascone, alpha-eudesmol, alpha-funebrene, alpha-guaiene, alpha-gurjunene, alpha-humulene, alpha-santalene, alpha-santalol, alpha-selinene, alpha-sinensal, alpha-terpineol, alpha-terpinolene, alpha-vetivone, ambroxan/ambrein, amorphadiene, aristolene, aromadendrene, artemisinic acid, asiatic acid, astaxanthin, atisane, bergamotene, beta pinene, beta-bisabolene, beta-bisabolol, beta-carotene, beta-caryophyllene, beta-Damascone, beta-eudesmol, beta-guaiene, beta-santalene, beta-santalol, beta-sinensal, beta-thujone, beta-vetivenene, beta-vetivone, beta-Ylangene, bisabolol, boswelic acid, camphene, camphor, carvacrol, carveol, carvone, caryophyllene oxide, cedrenes, celastrol, cembrene, ceroplastol, cineol, cis-abienol, citral, citronellal, citronellol, copalol, cubebol, cucurbitane, cyperene, cyperene epoxide, cyperotundone, damascenone, dehydrofukinone, delta-cadinene, delta-damascone, delta-guaiene, delta-selinene, dihydro agarofuran, E-alpha-bisabolene, E-gamma-bisabolene, eleutherobin, epi-b-santalol, epi-zizaene, epi-zizaenone, eugenol, evopimaradene, farnescene, farnesol, fenchone, forskolin, gamma-bisabolol, gamma-cadinene, gamma-eudesmol, gamma-gurjunene, gamma-humulene, gamma-muurolene, gamma-terpinene, gascardic acid, geraniol, geranylgeraniol, germacrene D, glycyrrhizin, guaiol, haslene, ionones, iripallidal, irones, isoborneol, isopemaradiene, isoprene, iso-velencenol, jinkohols, karanone, kaurene, kessane, khusimene, khusimol, labdenediol, ledene, ledol, levopimaradiene, levopimaric acid, linalool, linalool oxide, longifolenaldehyde, longipinene, L-rose oxide, lupeol, madeccasic acid, menthol, menthone, methyl vetivenate, mogrosides, muurolenes, myrcene, nerolidol, nootkatene, nootkatol, nootkatone, ocimenes, ophiobolin A, patchouli alcohol, pinene, piperitone, pogostol, prenol, protopanaxadiol, protopanaxatriol, pulegone, R-(−)-carvone, rebaudioside D, rebaudioside M, rotundone, S-(+)-carvone, sabinene, sabinene hydrate, santalals, santalenes, santalols, sclarene, sclareol, selina-3,7(11)-diene, selinadiene, spathulenol, steviol, steviol glycosides, sulcatone, tagetone, taxadiene, thymol, ursolic acid, valencene, valeranone, verbenone, vetiverol, vetiverone, vetiveryl acetate, viridiflorol, Z,E-alpha-bergamotol, Z-alpha-bisabolene, zeaxanthin, Z-gamma-bisabolene, zizaene, zizenone, and Z-lanceol, including isoforms and derivatives thereof. In some embodiments, the secondary metabolite is a monoterpenoid, diterpenoid, sesquiterpenoid, or triterpenoid.

As described in U.S. Pat. No. 8,927,241, terpene or terpenoid products include: artemisinin; taxol; taxadiene; levopimaradiene; gingkolides; abietadiene; abietic acid; beta-amyrin; retinol; thymoquinone; ascaridole; beta-selinene; 5-epi-aristolochene; vetispiradiene; epi-cedrol; alpha, beta and y-humulene; a-cubebene; beta-elemene; gossypol; zingiberene; periplanone B; capsidiol; capnellene; illudin; isocomene; cyperene; pseudoterosins; crotophorbolone; englerin; psiguadial; stemodinone; maritimol; cyclopamine; veratramine; aplyviolene; macfarlandin E; betulinic acid; oleanolic acid; ursoloic acid; pimaradiene; neo-abietadiene; squalene; dolichol; lupeol; euphol; kaurene; gibberellins; cassaic acid; erythroxydiol; trisporic acid; podocarpic acid; retene; dehydroleucodine; phorbol; cafestol; kahweol; tetrahydrocannabinol; androstenol; or a derivative thereof.

In some embodiments, the terpene or terpenoid product is squalene, or a derivative thereof (e.g., squalane). Squalene is a triterpenoid sometimes obtained from shark liver oil. Alternative sources include vegetable oils, such as olive oil. All plants and animals produce squalene as a biochemical intermediate, and squalene is the biochemical precursor to steroids. Oxidation (via squalene monooxygenase) of one of the terminal double bonds of squalene yields 2,3-squalene oxide, which undergoes enzyme-catalyzed cyclization to lanosterol, which is then converted to cholesterol and other steroids. Squalene has utility as a dietary supplement, adjunctive therapy in cancer, in vaccine adjuvants, as well as a component in skin and hair care products, for example. Squalene is toxic to microbial cells, and thus its synthesis by fermentation processes can be enhanced by extraction and/or sequestration of product from the culture.

In some embodiments, the terpenoid is a steviol glycoside (e.g., rebaudioside M), mogroside (e.g., Mogroside V), or comprises one or more of valencene, nootkatol (α and/or β), and nootkatone. Exemplary enzymatic pathways are disclosed in US 2015/0322473 and WO 2016/050890 (mogroside), US 2017/0332673 (steviol glycosides), and US 2018/0135081 (valencene, nootkatol, and/or nootkatone), which are each hereby incorporated by reference in their entireties.

In some embodiments, the secondary metabolite is a cannabinoid. Exemplary metabolic pathways for biosynthesis of cannabinoids is described in WO 2016/010827 and U.S. Pat. No. 9,822,384, which are hereby incorporated by reference in their entireties.

In some embodiments, the secondary metabolite is a polyketide. Exemplary metabolic pathways for biosynthesis of polyketides are described in WO 2017/160801, which is hereby incorporated by reference in its entirety.

Bacterial host cells can be engineered for increased carbon flux through the MEP pathway. Exemplary genetic modification to increase MEP carbon are disclosed in US 2018/0245103 and US 2018/0216137, which are hereby incorporated by reference in their entireties. In various embodiments, the microbial cell overexpresses one or more enzymes in the MEP pathway (for bacterial host cells) or MVA pathway (for yeast host cells), including by enzyme duplication, or engineering enzymes for increased activity or expression.

In various embodiments, the secondary metabolite is produced by the microbial cell through one or more enzymatic steps, which may comprise an oxygenation, glycosylation, and/or a prenyl transferase reaction.

In some embodiments, the microbial host expresses one or more recombinant oxygenase enzymes, which decorate the metabolite core with one or more hydroxyl, aldehyde or ketone groups. In some embodiments, the cell produces a blend of target metabolites with varying levels of oxygenation (e.g., valencene, nootkatol, and nootkatone). In such embodiments, the composition and relative amount of the extraction phase impacts the titer of oxygenated product, and can therefore be tuned to impact yield or relative amount of target products. In some embodiments, the oxygenase is selected from a cytochrome P450 (CYP450) enzyme, a non-heme iron oxidase, or a laccase enzyme. CYP450 enzymes are involved in the formation (synthesis) and breakdown (metabolism) of various molecules and chemicals within cells. Recombinant expression of P450 enzymes in E. coli, including with respect to engineered membrane anchors, are described in US 2018/0251738, which is hereby incorporated by reference in its entirety. The CYP450 enzyme requires the presence of an electron transfer protein capable of transferring electrons to the CYP450 protein. In some embodiments, this electron transfer protein is a cytochrome P450 reductase (CPR), which can be expressed by the microbial host cell. In some embodiments, the oxygenase enzyme is a non-heme iron oxygenase (NHIO) or a laccase.

In some embodiments, the synthesis of the secondary metabolite includes at least one oxygenation reaction, and optionally, includes at least 2, at least 3, at least 4, or at least 5 oxygenation reactions, which can optionally be performed by one or a plurality (e.g., 2, 3, 4, or 5) of oxygenase enzymes (e.g., P450 enzymes).

In some embodiments, the microbial cell expresses one or more glycosyl transferase enzymes, producing a glycosylated secondary metabolite. For example, the microbial cell may express one or more UDP-dependent glycosyl transferase enzymes (UGT enzymes). UGT enzymes for glycosylation of terpenoid (e.g., steviol) glycosides (including for biosynthesis of RebM) are disclosed in US 2017/0332673, which is hereby incorporated by reference in its entirety. Other UGT enzymes are disclosed in WO 2018/031955, US 2017/0321238, and U.S. Pat. No. 9,920,349, which are hereby incorporated by reference in their entireties.

In some embodiments, synthesis of the secondary metabolite includes at least one glycosylation reaction, and optionally at least 2, at least 3, at least 4, at least 5, or at least 6 glycosylation reactions. The glycosylation reactions can be performed by one or a plurality (e.g., 2, 3, 4, or 5) of glycosyltransferase enzymes (e.g., UGT enzymes).

In some embodiments, the microbial cell expresses one or more recombinant prenyl transferase enzymes, producing a prenylated metabolite. In some embodiments, the prenyl transferase enzyme is a geranyl diphosphate synthase (GPPS), a farnesyl diphosphate synthase (FPPS), or a geranylgeranyl diphosphate synthase (GGPPS). Exemplary enzymes are disclosed in US 2017/0332673 and US 2018/0135081, which are hereby incorporated by reference in their entireties.

The microbial cell is grown in an aqueous phase in a bioreactor. The microbial cell may be cultured in batch culture, continuous culture, or semi-continuous culture. In some embodiments, the microbial cell is cultured using a fed-batch process comprising a first phase where bacterial biomass is created, followed by a secondary metabolite production phase. Fed-batch culture is a process where nutrients are fed to the bioreactor during cultivation and in which the product(s) remain in the bioreactor until the end of the run. Generally, a base medium supports initial cell culture and a feed medium is added to prevent nutrient depletion. The controlled addition of the nutrient directly affects the growth rate of the culture and helps to avoid overflow metabolism and formation of side metabolites.

The aqueous phase generally comprises an appropriate cell culture medium, including initial culture medium and feed medium, for the host cells. An exemplary batch media for growing the microbial cells (producing biomass) comprises, without limitation, yeast extract. During the biosynthesis phase, the aqueous phase may further comprise precursor molecules for production of the secondary metabolite. For example, in some embodiments, the microbial cell synthesizes the secondary metabolite from basic carbon substrates (e.g., C1-C6 carbon substrates), such as glucose or glycerol. In some embodiments, carbon substrates such C1, C2, C3, C4, C5, and/or C6 carbon substrates are fed to the culture for production of the target product, e.g., with carbon flux through the MEP or MVA pathway or other metabolic pathway. In exemplary embodiments, the carbon source is glucose, sucrose, fructose, xylose, and/or glycerol.

In other embodiments, the microbial cells are fed product precursors, which may be substrates for synthetic enzymes, and/or substrates for glycosylation, oxygenation, or prenylation, or transfer of other chemical groups or moieties to a core structure. For example, the precursor can be a terpene or terpenoid compound that is a substrate for one or more synthetic enzymes, oxygenation reaction, and/or glycosylation reactions.

In various embodiments, host cells can be cultured under aerobic, microaerobic, or anaerobic conditions. In some embodiments, the culture is maintained under aerobic conditions, or microaerobic conditions. For example, when using a fed-batch process, the biomass production phase can take place under aerobic conditions, followed by reducing the oxygen levels for the product production phase. For example, the culture can be shifted to microaerobic conditions after from about 10 to about 20 hours. In this context, the term “microaerobic conditions” means that cultures are maintained just below detectable dissolved oxygen. See, Partridge J D, et al., Transition of Escherichia coli from Aerobic to Micro-aerobic Conditions Involves Fast and Slow Reacting Regulatory Components, J. Biol. Chem. 282(15):11230-11237 (2007). In some embodiments, optimum oxygen levels during the production phase are empirically determined.

The production phase includes feeding a nitrogen source and a carbon source. For example, the nitrogen source can comprise ammonium (e.g., ammonium hydroxide). The carbon source may contain C1, C2, C3, C4, C5, and/or C6 carbon sources, such as, in some embodiments, glucose, sucrose, or glycerol. The nitrogen and carbon feeding can be initiated when a predetermined amount of batch media is consumed, a process that provides for ease of scaling. The optimum carbon:nitrogen ration may be empirically determined. In some embodiments, the nitrogen feed rate is from about 8 L per hour to about 20 L per hour, but will depend in-part on the product, strain, and scale.

In various embodiments, the host cell may be cultured at a temperature between 22° C. and 37° C. While commercial biosynthesis in bacteria such as E. coli can be limited by the temperature at which overexpressed and/or foreign enzymes are stable, recombinant enzymes may be engineered to allow for cultures to be maintained at higher temperatures, resulting in higher yields and higher overall productivity. In some embodiments, the culturing is conducted at about 22° C. or greater, about 23° C. or greater, about 24° C. or greater, about 25° C. or greater, about 26° C. or greater, about 27° C. or greater, about 28° C. or greater, about 29° C. or greater, about 30° C. or greater, about 31° C. or greater, about 32° C. or greater, about 33° C. or greater, about 34° C. or greater, about 35° C. or greater, about 36° C. or greater, or about 37° C. In some embodiments, the culture is maintained at a temperature of from 22 to 37° C., or a temperature of from 25 to 37° C., or a temperature of from 27 to 37° C., or a temperature of from 30 to 37° C.

The extraction phase is added to the culture, at least during the biosynthesis phase, and can be an organic overlayer that sequesters the secondary metabolite for recovery, in addition to enhancing biosynthesis and extracellular transport. The extraction phase is predominately composed of substantially non-volatile compounds at bioreactor conditions. Components of the extraction phase will generally be liquid at fermentation conditions and have a boiling point above 150° C., or between 150 and 500° C., or between 200 and 400° C.

In some embodiments, the extraction phase comprises (or predominately comprises) one or more members selected from: medium, long chain, or cyclic hydrocarbon(s); plant or vegetable oil or components thereof; fatty acid glyceride(s), and fatty acid ester(s). In some embodiments, the extraction phase comprises or is predominately composed of a linear or branched hydrocarbon, optionally having from 10 to 24 carbon atoms, or from 12 to 18 carbon atoms. In some embodiments, the hydrocarbon is a linear hydrocarbon, and optionally comprises one or more double bonds, and optionally from 1 to 4 double bonds. In some embodiments, the extraction phase predominately comprises a hydrocarbon that is saturated or unsaturated, and may optionally comprise a cyclic group, which may be aromatic.

Where the extraction phase comprises or predominately comprises one or more saturated, mono-unsaturated, and/or polyunsaturated fatty acids, the fatty acids are optionally fatty acid esters (e.g., alkyl esters of fatty acids). In some embodiments, the fatty acids or fatty acid esters have from 12 to 24 carbon atoms.

In various embodiments, the extraction phase comprises one or a mixture of an ester of a fatty acid and alcohol, especially in cases where the product is a liquid oil (e.g., squalene) during fermentation. Esters of fatty acids can be methyl esters, ethyl esters, propyl esters, isopropyl esters, butyl esters, or isobutyl esters, among others. In some embodiments, the extraction phase comprises (or comprises a significant amount of) methyl esters of fatty acids (FAMEs) such as methyl decanoate, methyl laurate, methyl myristate, methyl palmitate, methyl stearate, and methyl oleate. In some embodiments, the extraction phase comprises (or predominately comprises) ethyl esters of fatty acids (FAEEs) such as ethyl decanoate, ethyl laurate, ethyl myristate, ethyl palmitate, ethyl stearate, and ethyl oleate. In this context, a significant amount is at least 10% or at least 20%, or at least 50% of the extraction phase. In an exemplary embodiment, the extraction phase is predominately methyl oleate.

In some embodiments, the extraction phase comprises (or comprises a significant amount of) triglycerides (e.g., predominately of C16 and C18 fatty acids). In some embodiments, the triglycerides are homogenous triglycerides (same fatty acid side chains). In these or other embodiments, the triglycerides may comprise heterogeneous triglycerides (mixed fatty acid side chains). In still other embodiments, the extraction phase comprises or further comprises one or more mono- or di-glycerides.

In some embodiments, the extraction phase comprises one or more alkanes, generally alkanes of chain length greater than eight, such as nonane, decane, dodecane, etc. For example, the alkane may have from 8 to 20 carbon atoms, or from 8 to 16 carbon atoms is some embodiments. In some embodiments, the extraction phase comprises a blend of alkanes (e.g. mineral oil).

In some embodiments, the extraction phase comprises one or a blend of ionic liquids. An ionic liquid is a salt in which the ions are poorly coordinated, which results in these solvents being liquid below 100° C. Generally, at least one ion has a delocalized charge and one component is organic (such as an aromatic and/or heterocyclic ring system), which prevents the formation of a stable crystal lattice. Properties, such as melting point, viscosity, and solubility are determined by the substituents on the organic component and by the counterion. An exemplary ionic liquid is 1-butyl-3-methylimidazolium hexafluorophosphate (CAS #174501-64-5).

In some embodiments, the extraction phase comprises a silicone oil or a blend of silicone oils. Silicone oils comprise polymerized siloxane with organic side chains, such as polydimethylsiloxane. Silicone oils are primarily used as lubricants, and some have advantageous anti-foaming properties due to their low surface tension. In some embodiments, the silicone oil is cyclicsiloxane or is a non-cyclicsiloxane. In some embodiments, the silicone oil comprises simethicone, which has low surface tension and good anti-foaming properties.

In some embodiments, the extraction phase comprises a perfluorinated oils or a blend of perfluorinated oils, such as a perfluoropolyether oil. An exemplary perfluorinated oil is 3M Novec HFE-7500; 2-(Trifluoromethyl)-3-ethoxydodecafluorohexane (CAS #297730-93-9).

In some embodiments, the extraction phase comprises a blend of some (e.g., 2, 3, or 4) of the above classes, or all of the above classes (ester of a fatty acid and alcohol, triglyceride, ionic liquid, silicone oil, alkane/mineral oil, and perfluorinated oil). In these or other embodiments, the extraction phase is stabilized by a surfactant or emulsifier. Exemplary surfactants include compounds capable of reducing the surface tension of water and for the interfacial tension between water and an immiscible liquid (e.g., the extraction phase). Exemplary emulsifiers include non-ionic emulsifiers such as polyol esters (e.g. ethylene glycol, diethylene glycol, glycol stearate and propylene glycol monoesters of fatty acids), and glycerol esters (e.g. glyceryl stearate, glyceryl monooleate, glycerylmonolaurate, glyceryl ricinolate, glyceryl monocaprylate). Exemplary emulsifiers further include Sorbitan derivatives, which are esters of cyclic anhydrides of sorbitol with a fatty acid. These include sorbitan monolaurate, sorbitan monooleate, sorbitan monostearate, sorbitan monopalmitate, sorbitan sesquioleate, sorbitan trioleate, sorbitan tristearate, polyoxyethylene sorbitan esters (e.g. polyoxyethylene sorbitan monostearate, polyoxyethylene sorbitan tristearate, polyoxyethylene sorbitan monooleate). Exemplary emulsifiers also include polyoxyethylene esters, which are mixtures of mono- or di-fatty acid esters (from C12 to C18) of polyoxyethylene glycol.

In some embodiments, the extraction phase comprises solubilized fatty acids, including for example, short chain, medium chain, and long chain fatty acids.

In some embodiments, the extraction phase comprises or predominately comprises one or more plant oils or vegetable oils. For example, the plant or vegetable oil may be selected from one or more of coconut oil, palm oil, cottonseed oil, wheat germ oil, soybean oil, sesame oil, olive oil, corn oil, sunflower oil, safflower oil, peanut oil, flaxseed oil, grape seed oil, and rapeseed oil. In some embodiments, the extraction phase comprises safflower oil, or is substantially or predominately comprised of safflower oil. Safflower oil is predominately composed of triglycerides, with C16 and C18 chains (e.g., C16:0, C18:0, C18:1, C18:2, C18:3). In some embodiments, the extraction phase comprises triglycerides with linoleic and oleic acid tails.

The composition and relative amount of the extraction phase can impact the amount of product that is oxygenated, which can be due to an impact on aeration. In some embodiments, at least 25% or at least 75% of recovered secondary metabolite is oxygenated product. In some embodiments, from 25% to about 75% of recovered secondary metabolite is oxygenated product, providing a blend of product at different levels of oxygenation. Such products can provide unique sensory characteristics, which are of particular value for the perfume and flavor industries.

The extraction can be designed to impact selective extracellular export of product, providing for greater yield, or in some embodiments, providing for a blend of product and intermediate. Such blends can provide unique biological or sensory properties. In some embodiments, the extraction phase recovers at least a 2:1 ratio of a secondary metabolite to an intermediates. In this context, the term “intermediate” includes compounds sharing a core structure or class of molecule with the target compound, such as a terpene or cannabinoid. In some embodiments, the extraction phase recovers at least a 5:1 ratio of the secondary metabolite to intermediates, or at least a 10:1 ratio of the secondary metabolite to intermediates.

When using extraction phases of less than 10% (v/v) with respect to the aqueous phase, after recovery of the extraction phase, the extraction phase can contain a high mass of the secondary metabolite (the product(s)). In some embodiments, the mass of product recovered is higher than with the use of a 10% dodecane overlayer. In various embodiments, after the production phase of the culture, the secondary metabolite is at least about 10% of the extraction phase by weight, or is at least about 20% of the extraction phase by weight, or is at least about 50% of the extraction phase by weight.

The secondary metabolite is recovered from the extraction phase, and product optionally isolated by any suitable process. In some embodiments, the product is purified by sequential extraction and purification. For example, the product may be purified by chromatography-based separation and recovery, such as supercritical fluid chromatography. The product may be purified by distillation, including simple distillation, steam distillation, fractional distillation, wipe-film distillation, or continuous distillation.

The production of the desired product can be determined and/or quantified, for example, by gas chromatography (e.g., GC-MS). Production of product, recovery, and/or analysis of the product can be done as described in US 2012/0246767, which is hereby incorporated by reference in its entirety. For example, in some embodiments, product oil is extracted from aqueous reaction medium using the extraction phase, followed by fractional distillation. In other embodiments, product oil is extracted from aqueous reaction medium using a hydrophobic extraction phase material, such as a vegetable oil, followed by organic solvent extraction and fractional distillation. Components of fractions may be measured quantitatively by GC/MS, followed by blending of fractions to generate a desired product profile.

In various embodiments, the recovered secondary metabolite product is incorporated into a consumer or industrial product. For example, the product may be a flavor product, a fragrance product, a sweetener, a cosmetic, a cleaning product, a detergent or soap, or a pest control product. Thus, the invention further provides methods of making products such as foods, beverages, texturants (e.g., starches, fibers, gums, fats and fat mimetics, and emulsifiers), pharmaceutical products, dietary supplements, tobacco products, nutraceutical products, oral hygiene products, skin and hair care products, and cosmetic products, by incorporating secondary metabolites produced herein. The higher yields of such species produced in embodiments of the invention can provide significant cost advantages as well as sustainability.

### EXAMPLES

### Example 1

### Composition of Extractive Phase

Microbial production of natural products relies on product transport to the extracellular environment. In addition, the extracellular milieu should prevent product degradation, evaporation, air stripping, and provide ease of separation and recovery. Experiments were conducted to determine the effect of different extractive phases on production of natural products in microbial culture, and evaluate whether the extractive phase can provide more than simple sequestration and separation advantages.

Historically and by convention, a 10% overlayer has been used throughout industrial and academic experiments when conducting microbial fermentations of volatile natural products. We hypothesize that the composition of the fermentation media/extractive phase emulsion have the potential to impact productivity in several ways, beyond simple compound sequestration. We therefore evaluated different oil compositions and percentage with respect to the aqueous phase in small scale fermentation.

In the first set of experiments, we investigated the use of alternative extractive phases in small scale fermentations: dodecane (a simple 12 carbon alkane), safflower oil (a much more complex mixture of triglycerides, with mostly unsaturated oleic and linoleic fatty acid tails), isopropyl myristate, isopropyl palmitate, and methyl oleate. The chemical properties of the alternative extractive phases are shown in Table 1:

TABLE 1

| Overlay | Chemical formula | BP | MW (g/mol) |
|---|---|---|---|
| Dodecane | C₁₂H₂₆ | 216° C. | 170.34 |
| Safflower oil | Triglycerides, with: | | ~800-900 |
| | C16:0 (~5.5%) | | |
| | C18:0 (~1.6%) | | |
| | C18:1 (~11.1%) | | |
| | C18:2 (~81.4%) | | |
| | C18:3 (~0.4%) | | |
| Isopropyl Myristate | C₁₇H₃₄O₂ | 315° C. | 270.45 |
| Isopropyl Palmitate | C₁₉H₃₈O₂ | 340° C. | 298.50 |
| Methyl Oleate | C₁₉H₃₆O₂ | 350° C. | 296.50 |

Alternative extractive phase were selected to test with an E. coli strain (“valencene strain”). Valencene strains produce valencene and oxygenated products of valencene. The valencene is the substrate for another enzyme that converts it to various oxygenated products. We selected the above alternative extractive phases due to their general similarity in physical properties and chemical structure to safflower oil, as well as their safety and availability.

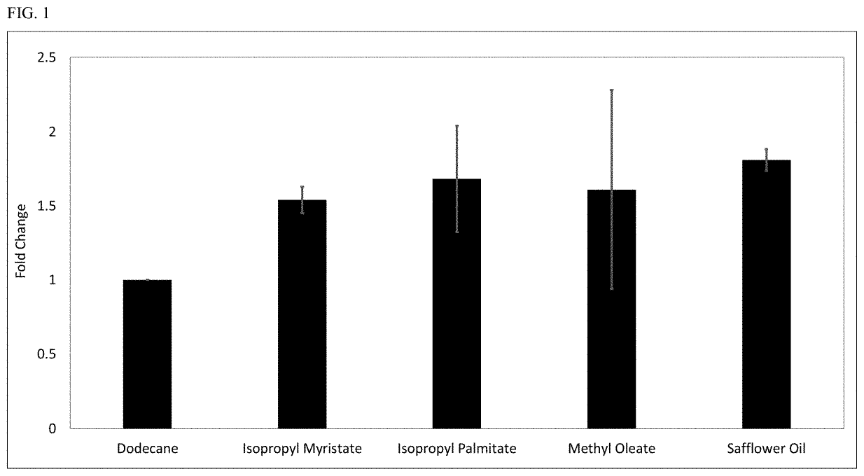

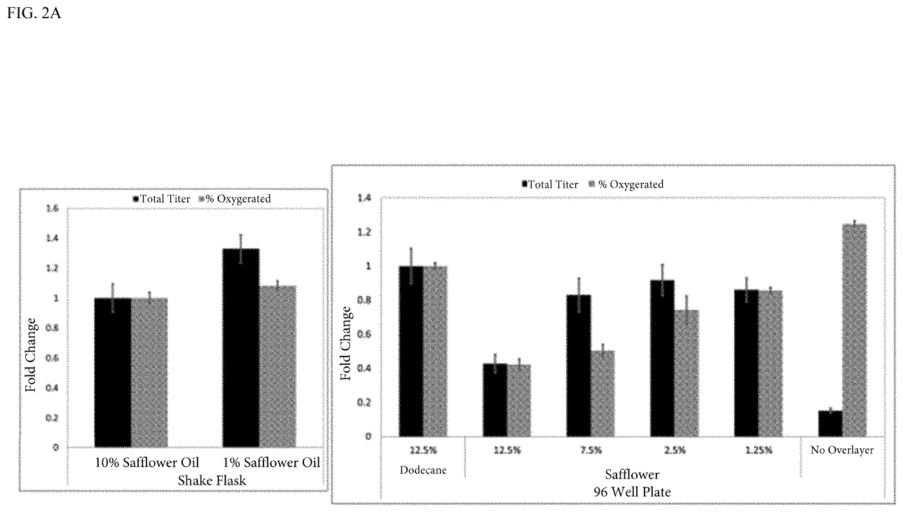

In FIG. 1, alternative extractive phases were tested for their suitability in small scale 96 well plate microbial fermentation of valencene strains. The goal was to maximize the product titer and conversion from valencene to oxygenated products. Only the non-oxygenated valencene titers are shown in FIG. 1.

In terms of valencene production the alternative extractive phases behave more similarly to safflower oil than dodecane. For a given extractive phase other than dodecane, valencene production is generally higher. Thus, the type of overlayer has a significant impact on productivity.

### Example 2

### Varying Overlayer Percentage

We hypothesized that, by changing the % extractive phase as well as the corresponding agitation, productivity of the fermentations could be improved with conventional dodecane or safflower extractive phases. The following example shows variation of safflower oil percent extractive phase (with respect to the aqueous phase). In addition, exemplary shake flask experiments were conducted.

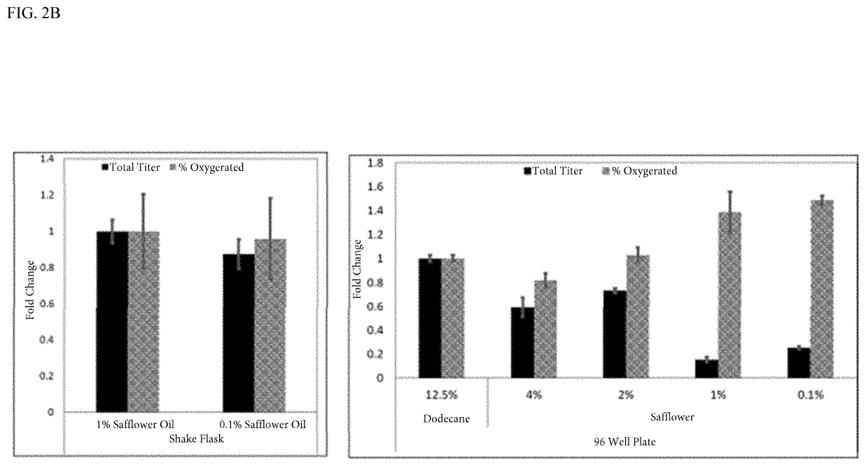

As shown in FIGS. 2A and B, lower safflower oil percentage improves both overall fermentation productivity as well as conversion to oxygenated products under appropriate conditions. This observation holds true for both 96 well plates and shake flasks. These data demonstrate that less overlayer can significantly improve productivity, which indicates there are likely phenomena beyond product capture and prevention of evaporation at play, as one would expect titers to decrease or stay the same as oil % is reduced, if the overlayer was entirely involved in capture and prevention of evaporation.

In addition, it is possible that reduced transport of valencene into the extracellular space increases intracellular residence time allowing additional chemistry to occur converting it to oxygenated products.

As shown in FIG. 2A, there is a strong and condition independent inverse trend between the % oxygenated product versus % overlayer. While evaporation certainly plays a role, clearly conversion and total titer between the 7.5% and 2.5% conditions across all plates and RPMs is improved, indicating evaporation and air stripping are not the only factors influencing productivity. Mass transfer and oxygenation are likely important factors. The shake flask experiment also shows improved % oxygenated conversion and higher overall titers between 10% vs. 1% safflower overlay.

In FIG. 2B, lower % safflower extractive phase is evaluated, given the results shown in FIG. 2A. The results confirm that there is a strong and condition independent inverse trend between the % oxygenated product versus % overlayer. Oxygenated percentage is increased to 90% with reduced safflower oil, albeit at the expense of titer in 96 well plates. Interestingly, in shake flasks there is not dramatic a drop in titer for 0.1% oil (as compared to 1%). Interestingly, 0.1% oil is ˜1 g/L, which means that the resulting extraction phase is operating at 50/50 product to safflower oil ratio.

### Example 3

### Bioreactor Experiments

In this Example, we evaluate how well the reduction in safflower oil percentage scales to a bioreactor experiment. We ran 2 Sartorius 2 L fed batch bioreactors with either 1% or 10% safflower oil to determine the effects on cell growth and productivity.

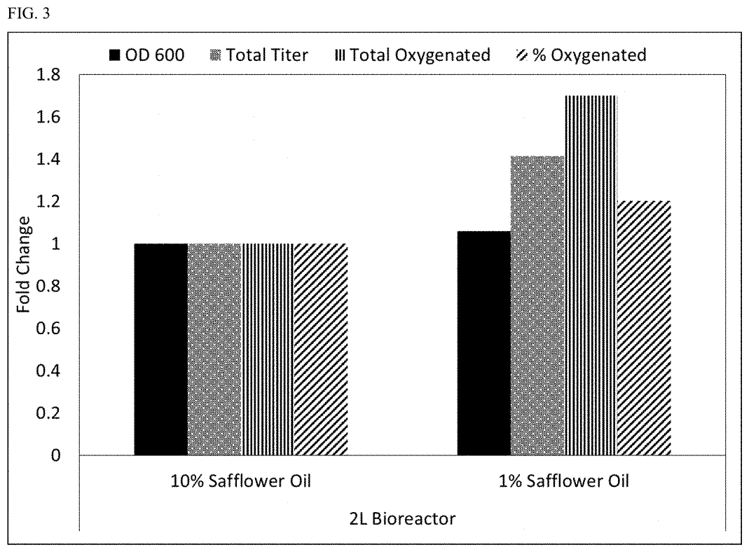

As shown in FIG. 3, the bioreactor data correlate more with the shake flask data than 96 well plate data. We see an increase in the % oxygenated conversion (bottom, right panel) with an increase in overall titer. The total oxygenated production with 1% safflower extractive phase is significantly higher. Microbial growth is similar in the two conditions.

Extraction phases, such as safflower oil, and the % with respect to the aqueous phase, are important variables for microbial production of secondary metabolites, such as terpenoids. The composition and % of the extraction phase impacts overall productivity and selectivity for different products.

Patents and patent publications cited herein are hereby incorporated by reference in their entireties.

1. A method for producing a secondary metabolite in microbial culture, comprising:
culturing a microbial cell producing a secondary metabolite in a bioreactor medium, the medium comprising an aqueous phase and an extraction phase, wherein the extraction phase enhances production of the secondary metabolite from microbial cells and/or enhances extracellular transfer of the metabolite.

2. (canceled)

3. The method of claim 1, wherein the composition of the extraction phase and relative amount of the extraction phase relative to the aqueous phase (% vol.) produce at least a 10% increase in the amount of the secondary metabolite in the extraction phase, as compared to a 10% (v/v) overlay of dodecane.

4. (canceled)

5. (canceled)

6. (canceled)

7. (canceled)

8. The method of claim 1, wherein the relative amount of the extraction phase in the bioreactor relative to the aqueous phase is between 0.1% and 8% (v/v).

9. (canceled)

10. (canceled)

11. The method of claim 1, wherein the volume of the aqueous phase in the bioreactor is at least about 1,000 L L.

12. The method of claim 1, wherein the microbial cell is a bacterium.

13. (canceled)

14. (canceled)

15. The method of claim 12, wherein the bacteria is E. coli.

16. The method of claim 1, wherein the microbial cell is a yeast cell, which is optionally an oleaginous yeast cell.

17. (canceled)

18. The method of claim 1, wherein the secondary metabolite is a terpene, terpenoid, alkaloid, cannabinoid, steroid, saponin, glycoside, stilbenoid, polyphenol, flavonoid, antibiotic, polyketide, fatty acid, or peptide.

19. (canceled)

20. The method of claim 18, wherein the metabolite is squalene or a derivative thereof, and the host cell is optionally E. coli or an oleaginous yeast.

21. The method of claim 1, wherein the secondary metabolite is produced by the microbial cell through one or more enzymatic steps selected from an oxygenation, glycosylation, and/or a prenyl transferase reaction.

22. The method of claim 1, wherein the microbial cell synthesizes the secondary metabolite from primary metabolites, or precursor molecules added to the culture.

23. The method of claim 22, wherein the microbial cell overexpresses one or more enzymes in the MEP or MVA pathway.

24. (canceled)

25. The method of claim 21, wherein synthesis of the secondary metabolite includes at least one oxygenation reaction.

26. (canceled)

27. (canceled)

28. (canceled)

29. (canceled)

30. (canceled)

31. (canceled)

32. The method of claim 1, wherein the extraction phase comprises one or more members selected from: medium, long chain, or cyclic hydrocarbon(s); plant or vegetable oil or components thereof; fatty acid glyceride(s), and fatty acid ester(s).

33. (canceled)

34. The method of claim 32, wherein the extraction phase comprises one or more saturated, mono-unsaturated, and/or polyunsaturated fatty acids, where the fatty acids are optionally fatty acid esters.

35. The method of claim 34, wherein the extraction phase comprises esters of fatty acids, and which are optionally selected from methyl esters, ethyl esters, propyl esters, isopropyl esters, butyl esters, and isobutyl esters.

36. The method of claim 34, wherein the extraction phase predominately comprises methyl oleate, isopropyl myristate, isopropyl palmitate, and/or safflower oil.

37. (canceled)

38. (canceled)

39. The method of claim 1, the extraction phase comprises one or more of a mineral oil, one or a blend of ionic liquids, a silicone oil or a blend of silicone oils, and one or a blend of perfluorinated oils.

40. (canceled)

41. (canceled)

42. (canceled)

43. The method of claim 32, wherein the extraction phase is stabilized with a surfactant.

44. (canceled)

45. The method of claim 32, wherein the extraction phase comprises one or more plant oils or vegetable oils.

46. (canceled)

47. The method of claim 45, wherein the plant or vegetable oil is safflower oil.

48. The method of claim 1, wherein after recovery of the extraction phase, the secondary metabolite is at least about 10% of the extraction phase by weight.

49. (canceled)

50. (canceled)

51. (canceled)

52. (canceled)

53. (canceled)

54. (canceled)

55. The method of claim 1, wherein the secondary metabolite is recovered from the extraction phase.

56. A method for preparing a consumer or industrial product, comprising: incorporating the secondary metabolite produced according to the method of claim 1, into the consumer or industrial product.

57. (canceled)

58. (canceled)
